(19) United States
(12) Patent Application Publication
(10) Pub. No.: US 2019/0066383 A1
(43) Pub. Date: Feb. 28, 2019

(54) METHOD AND SYSTEM FOR PERFORMING VIRTUAL-REALITY-BASED ASSESSMENT OF MENTAL AND BEHAVIORAL CONDITION

(71) Applicant: National Taiwan Normal University, Taipei City (TW)

(72) Inventors: Tzu-Chien LIU, Taipei City (TW); Li-Yu HUNG, Taipei City (TW); Yu-Chen KUO, Taipei City (TW)

(73) Assignee: National Taiwan Normal University, Taipei City (TW)

(21) Appl. No.: 16/101,720

(22) Filed: Aug. 13, 2018

(30) Foreign Application Priority Data
Aug. 25, 2017 (TW) 106128879

(51) Int. Cl. G06T 19/00; A61B 5/16; G06K 9/00; G02B 27/01; G06F 3/01

(52) U.S. Cl. CPC G06T 19/006; A61B 5/165; G06F 3/013; G02B 27/0172; G06K 9/00335

(57) ABSTRACT

A method for performing an assessment of mental and behavioral condition of a subject includes displaying, by a virtual reality (VR) device, a VR scene; generating, by the VR device, a notification indicating an assignment for the subject in the VR scene; recording, by a recording device, a response of the subject to the assignment; calculating, by a processing device, at least one score based on the response; and comparing, by the processing device, the score with a pre-established normative scale to generate an assessment result for the mental and behavioral condition of the subject.

### CROSS-REFERENCE TO RELATED APPLICATION

This application claims priority of Taiwanese Patent Application No. 106128879, filed on Aug. 25, 2017.

### FIELD

The disclosure relates to a method and a system for performing a virtual-reality-based assessment, and more particularly to a method and a system for performing a virtual-reality-based assessment of mental and behavioral condition.

### BACKGROUND

A conventional way for performing a mental assessment is to use a questionnaire, to present the same orally, on paper or a display, and to scale the responses to the questionnaire. For example, in order to determine whether a subject has attention deficit hyperactivity disorder (ADHD), a number of questions may be taken from the Diagnostic and Statistical Manual of Mental Disorders, fifth edition (DSM-5) to determine whether the subject has signs such as “Have trouble completing or turning in homework assignments, often losing things (e.g., pencils, toys, assignments) needed to complete tasks or activities”, and “Seem to not be listening when spoken to”. The responses may be filled out by the subject himself/herself or by a third party based on an observation of the subject (e.g., a teacher to a student). After the mental assessment indicates that the subject may have ADHD, the subject may then be referred to a professional for treatment.

### SUMMARY

One object of the disclosure is to provide a method that can alleviate at least one of the drawbacks of the prior art.

According to one embodiment of the disclosure, the method includes:

displaying, by a virtual reality (VR) device, a VR scene;

generating, by the VR device, a notification indicating an assignment for the subject in the VR scene;

recording, by a recording device, a response of the subject to the assignment;

calculating, by a processing device, at least one score based on the response; and

comparing, by the processing device, the score with a pre-established normative scale to generate an assessment result for the mental and behavioral condition of the subject.

Another object is to provide a system that is capable of implementing the above-mentioned method.

According to one embodiment of the disclosure, the system includes a virtual reality (VR) device, a recording device, and a processing device communicating with the VR device and the recording device.

The processing device is programmed to:
control the VR device to display a VR scene, and to generate a notification in the VR scene, the notification indicating an assignment for the subject in the VR scene;
in response to the recording device recording a response of the subject to the assignment, calculating at least one score based on the response; and
comparing the score with a pre-established normative scale to generate an assessment result for the mental and behavioral condition of the subject.

### BRIEF DESCRIPTION OF THE DRAWINGS

Other features and advantages of the disclosure will become apparent in the following detailed description of the embodiments with reference to the accompanying drawings, of which:

FIG. 1 is a block diagram illustrating a system for performing an assessment of mental and behavioral condition for a subject according to one embodiment of the disclosure;

FIG. 2 is a flow chart illustrating steps of a method for performing an assessment of mental and behavioral condition of a subject according to one embodiment of the disclosure;

FIG. 3 illustrates an exemplary virtual reality (VR) scene according to one embodiment of the disclosure; and

FIG. 4 illustrates a manner for notifying the subject of a number of assignments.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

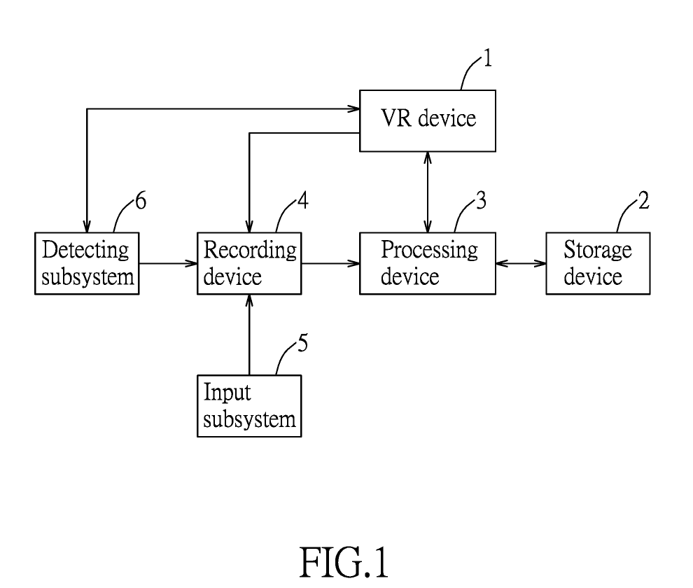

FIG. 1 is a block diagram illustrating a system for performing an assessment of mental and behavioral condition for a subject according to one embodiment of the disclosure.

The system includes a virtual reality (VR) device 1, a storage device 2, a processing device 3, a recording device 4, an input subsystem 5 and a detecting subsystem 6.

The VR device 1 may be embodied using a head mount display (HMD) or other suitable device that, when worn by a subject (e.g., a human) is capable of displaying a VR scene for the subject.

The storage device 2 may be embodied using a physical data storage device such as a hard disk (HD), a solid state drive (SSD), a flash drive, etc. The storage device 2 stores therein a software application, at least one script file and a plurality of virtual objects.

The processing device 3 may be embodied using a desktop computer, a laptop, a tablet, a mobile phone, or other electronic devices capable of performing processing operations. The processing device 3 is coupled to the VR device 1 and the storage device 2, and may be operated by a user to execute the software application, which includes instructions that, when executed by the processing device 3, cause the processing device 3 to perform a number of operations.

The recording device 4 may be embodied using an electronic device that is similar to the processing device 3. In one embodiment, the recording device 4 is embodied using a server device that communicates with the components of the system through a network (e.g., the Internet). In another embodiment, the storage device 2, the processing device 3 and the recording device 4 may be integrated in the form of a single electronic device.

The input subsystem 5 is coupled to the recording device 4, and may be embodied using a keyboard, a mouse, a joystick, a graphic tablet, a microphone, or a combination thereof.

The detecting subsystem 6 may include a gyroscope mounted on the VR device 1, an eye tracking device mounted on the VR device 1 or disposed near the subject, a heart rate (HR) monitor mounted on the subject, or a combination thereof. In particular, the gyroscope is for detecting head movement of the subject, the eye tracking device is for detecting a point of gaze of the subject, and the HR monitor is for measuring heart rate of the subject.

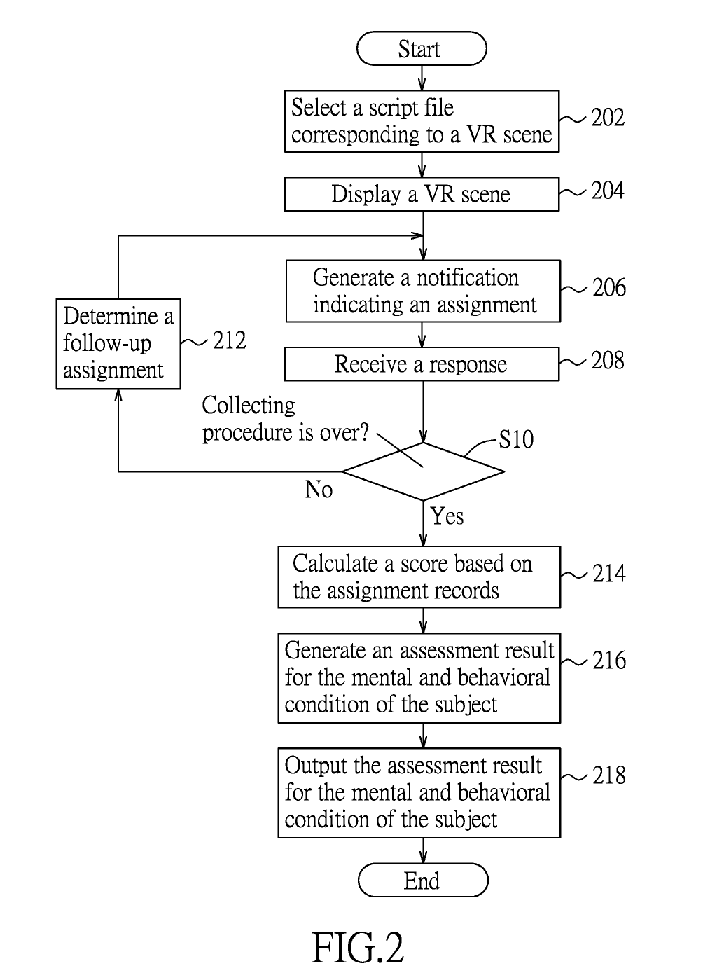

FIG. 2 is a flow chart illustrating steps of a method for performing an assessment of mental and behavioral condition of a subject that is implemented using the system of FIG. 1, according to one embodiment of the disclosure. In use, the processing device 3 may be operated by a conductor, the VR device 1 is worn by the subject, and the input subsystem 5 is operated by the subject.

In step 202, upon executing the software application, the processing device 3 displays an instruction for the conductor to select a script file corresponding to a VR scene to be presented to the subject. The conductor may also operate the processing device 3 to edit a script file stored in the storage device 2 or to create a new script file and store the new script file in the storage device 2. Each script file stores settings of a VR scene, and a plurality of virtual objects.

In this embodiment, the script file selected is associated with a questionnaire for determining whether the subject has attention deficit hyperactivity disorder (ADHD). The questionnaire is available in the Diagnostic and Statistical Manual of Mental Disorders, fifth edition (DSM-5). One condition related to ADHD is that the subject “frequently cannot stay focused doing homework or activities”, and as such, one VR scene may be created to have the subject actually do homework or activities in the VR scene. An exemplary VR scene may be a classroom, an office, a workplace, a playground, a ballpark, etc.

In step 204, the processing device 3 controls the VR device 1 to display the VR scene, so as to initiate a collecting procedure, in which the subject is to be presented with assignments and responses of the subject to the questions are to be collected.

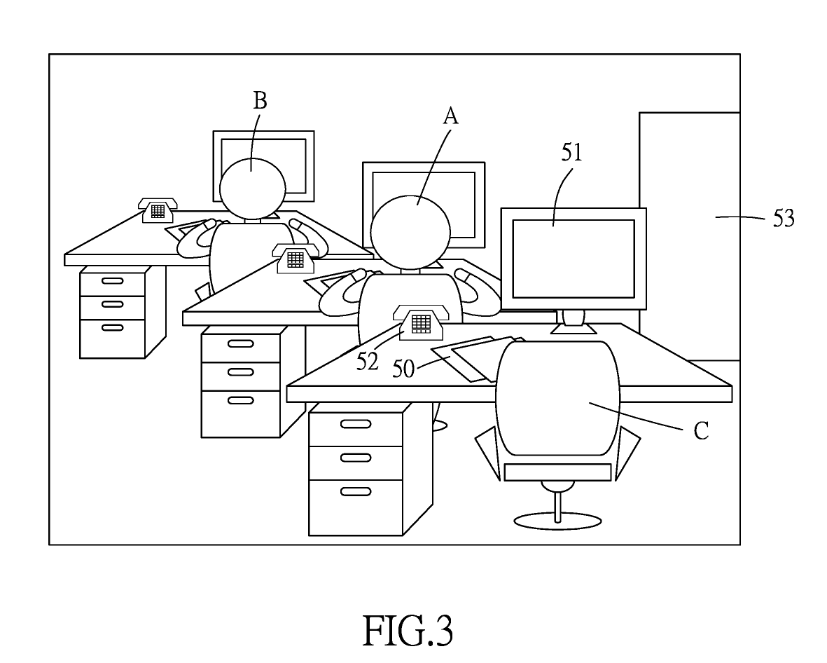

In one example, the VR scene is a part of an office (see FIG. 3), and a number of virtual objects that may be pre-stored in the storage device 2 may be included in the VR scene. As seen in FIG. 3, the virtual objects may include, but not limited to, a first avatar (A), a second avatar (B), a chair (C) to be sat by an avatar associated with the subject (not depicted in the drawings), papers 50, a display 51, a telephone 52, and a door 53.

It is noted that at least some of the virtual objects are programmed to permit interaction with the avatar of the subject. In this embodiment, the avatar of the subject may be a first-person perspective avatar, and thus any interaction between the virtual objects and the avatar of the subject can be considered as interaction with the subject. For example, when it is determined that the subject has been staring at one of the virtual objects for longer than a predetermined time threshold (based on detection result of the detecting subsystem 6), the one of the virtual objects may be “triggered”, producing a reaction (e.g., the first avatar (A) may speak to the subject). In another example, when the subject is interacting with the display 51, the display 51 may react by displaying a question (e.g., a math question) or a statement (e.g., a request to perform certain action), and the subject may operate components of the input subsystem 5 (e.g., the keyboard, the mouse) to input a response thereto.

When the VR scene is presented, in step 206, the processing device 3 controls the VR device 1 to generate a notification indicating an assignment for the subject in the VR scene based on the script file. Specifically, the notification may be in the form of a text bubble including texts instructing the subject to perform the assignment (e.g., complete a math question displayed on the papers 50 or the display 51, perform a specific action (e.g., speak), locate a specific object, spot an error in a paragraph of text, etc.). In other examples, the notification may be in the form of a vocal instruction (e.g., when the subject interacts with the telephone 52, the vocal instruction may be played). In one example, the notification may include both visual and audio elements. Specifically, as the vocal instruction is being played, the subject may be instructed to interact with a specific virtual object, or to perform a specific action upon hearing a particular sound.

In step 208, at least one of the input subsystem 5 and the detecting subsystem 6 receives a response from the subject to the assignment.

Specifically, based on the nature of the assignment, the response may be in the form of text or audio (which will be received by the input subsystem 5) or an action carried out by the subject (which will be received by the detecting subsystem 6). The response is then recorded by the recording device 4.

In one embodiment, the recording device 4 further records an attended-to position in the VR scene where the subject pays attention to during execution of the assignment, as detected by the detecting subsystem 6. For example, a point of gaze of the subject detected by the eye tracking device of the detecting subsystem 6 may serve as the attended-to position. In addition, the recording device 4 further records a degree of completion of the assignment (e.g., whether the subject has given a response, whether the question is correctly answered, etc.), which, along with the response and the attended-to position, are stored in the recording device 4 in an assignment record that is associated with the assignment.

In one embodiment, the recording device 4 or the detecting subsystem 5 may construct an attention circle with the point of gaze as a center, and determine a movement and coverage of the attention circle in the VR scene. Afterward, the recording device 4 records the movement and coverage of the attention circle in the assignment record. In use, the virtual objects in the VR scene may be configured to “attempt” to draw the user's attention. For example, in the VR scene of FIG. 3, the telephone 52 may ring, and the second avatar (B) may go to answer the telephone 52. After hanging up, the second avatar (B) may go on to talk to the first avatar (A). During the above events, the user's attention may be moving among the telephone 52, the second avatar (B) and the first avatar (A). Accordingly, the recording device 4 may record various information of the user's attention associated with each of the telephone 52, the second avatar (B) and the first avatar (A) (e.g., a duration and a frequency in which the user looks, a distance, a preset weight of the object, etc.).

In some embodiments, the assignment record may further record the virtual objects within the coverage of the attention circle, a number of the virtual objects within the coverage of the attention circle, a time instant when the subject attends to each of the virtual objects, and a time length for which the subject pays his/her attention to each of the virtual objects.

In one embodiment, the recording device 4 further records, in the assignment record, a length of time taken by the subject to complete the assignment in the assignment record (e.g., a length of time the subject takes to complete a math calculation, how fast the subject reacts to a task instruction, etc.).

In one embodiment, the recording device 4 further records, in the assignment record, a heart rate detected by the HR measuring device of the detecting subsystem 6. The recording device 4 may also record a heart rate distribution of the subject in a period of time in which the subject undertakes the assignment, in the assignment record.

By submerging the subject in the VR scene that is more closely related to a scene associated with the questions in the questionnaire, a more realistic and accurate response from the subject may be obtained for better assessment of mental and behavioral condition of the subject.

In one embodiment, in order to determine whether the subject is able to keep focus on the assignment, during the execution of the assignment by the subject in response to the notification generated in step 206, the processing device 3 may control the VR device 1, based on the script file, to generate at least one auxiliary notification in the VR scene for drawing attention of the subject away from performing the assignment. In the example of FIG. 3, the auxiliary notification may be in the form of the first avatar (A) assisting the subject in answering a question, the second avatar (B) trying to distract the subject by making a sound or walking by the avatar of the subject, the telephone 52 ringing, an object dropping of the table, or another avatar entering the VR scene through the door 53.

In such a case, the recording device 4 further records, in the assignment record, the movement and coverage of the attention circle and the HR distribution during the execution of the assignment by the subject, so as to serve as a basis for determining whether the subject is focused on the assignment or is distracted by the auxiliary notification (e.g., to determine a number of times the subject is distracted during the assignment).

In step 210, the processing device 3 determines whether the collecting procedure is over. In this embodiment, it is determined that the collecting procedure is over when one of a first condition that a predetermined number of assignments have been executed and a second condition that a predetermined length of time has elapsed is satisfied.

Specifically, the questionnaire may include twenty questions, and an assignment may be associated with a corresponding question of the questionnaire, and the collecting procedure is over when all twenty assignments are presented to the subject and the responses thereto are all collected. In some embodiments, the collecting procedure may be terminated after a predetermined length of time (e.g., 20 minutes) has elapsed since the initiation of the collecting procedure. In some embodiments, a particular response to a given assignment may require a further assignment be presented to collect additional response(s), thereby continuing the collecting procedure.

When it is determined in step 210 that the collecting procedure is over, the flow proceeds to step 214. Otherwise, the flow proceeds to step 212.

In step 212, the processing device 3 accesses the recording device 4 to obtain the assignment record that is recorded in step 208. Afterward, the processing device 3 determines a follow-up assignment based on the assignment record and an algorithm pre-stored in the script file.

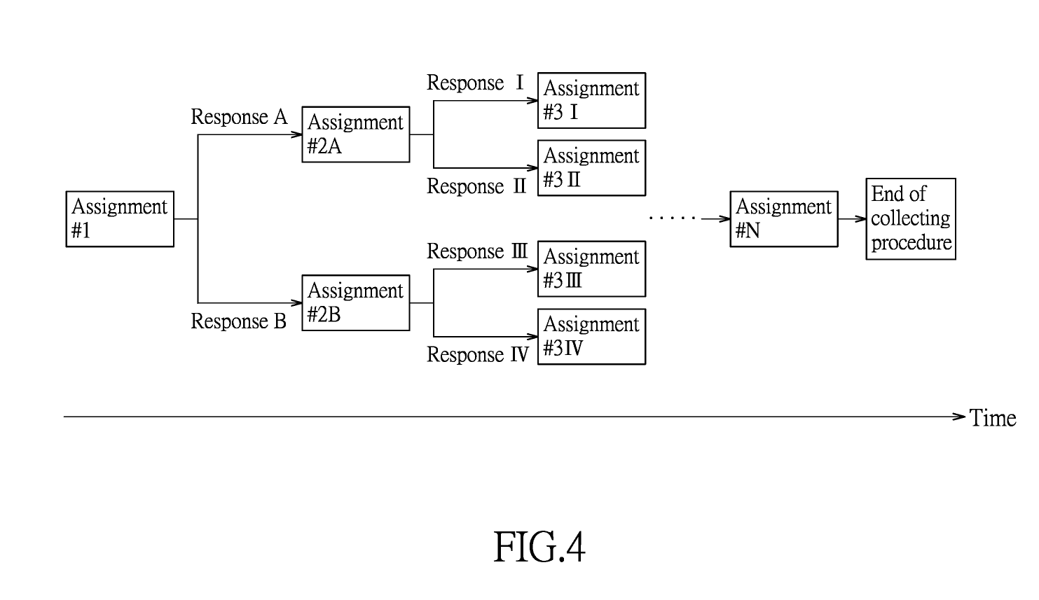

For example, as shown in FIG. 4, after an assignment #1 is finished, the processing device 3 obtains the assignment record for the assignment #1, in order to determine what assignment to present as an assignment #2. For example, different responses to the assignment #1 (categorized as A and B) may cause the processing device 3 to present different assignments as the assignment #2 (labeled as assignment #2A and assignment #2B, respectively). It is noted that some or all elements that are recorded in the assignment record as described above may be employed in the determination.

In the above-mentioned manner, since the follow-up assignment is determined based on the specific assignment record of the most-recently-undertaken assignment by the subject, the determination of the follow-up assignment is dedicated to the subject. Namely, even though two different subjects may carry out the same assignment initially, the follow-up assignments for the two subjects may be different since their responses to the same assignment may be different.

In one embodiment, the script file includes twenty notifications related to twenty assignments (numbered 1 to 20), and the processing device 3 may control the VR device 1 to present the notifications for the assignments in a non-recurring sequence (e.g., 1, 2, 3, . . . , 20). The non-recurring sequence may also be altered based on the assignment records (e.g., 1, 5, 7, . . . , 20).

In step 214, the processing device 3 calculates a score based on the assignment records of all assignments undertaken by the subject.

Afterward, in step 216, the processing device 3 compares the score with a pre-established normative scale to generate an assessment result for the mental and behavioral condition of the subject.

For example, the assessment is associated with determining how the subject is able to maintain attention in various functional components, such as alerting, orienting, executive attention, divided attention, etc. Accordingly, assignments may be associated with one or more functional components, and in step 214, a plurality of scores, each associated with one functional component, may be calculated.

The normative scale may be established by performing assessments on a particular population (e.g., a number of school kids in elementary schools located in a specific area), and creating the normative scale based on the assessments on the population.

In one example, after a score associated with the alerting function is calculated, the score may be compared with a normative scale as exemplified in the following Table 1.

TABLE 1

| | Alerting Attention Index | | | | |
|---|---|---|---|---|---|
| Description | Very Low | Low | Normal | High | Very High |
| Score | 0-10 | 11-15 | 16-20 | 21-25 | 26-30 |

It is noted that a subject having a score in the range of 11 to 15 may be considered to have a relatively low alerting function, a subject having a score in the range of 16 to 20 may be considered to have an average alerting function, and a subject having a score in the range of 21 to 25 may be considered to have a relatively high alerting function. The operation of step 216 is similar to a norm-referenced test (NRT).

In step 218, the processing device 3 outputs the assessment result for the mental and behavioral condition of the subject. Specifically, the assessment result may be displayed on a display of the processing device 3, or transmitted to a remote server over a network. Using the result, the conductor or other parties may determine whether the subject has one or more specific syndromes, and whether treatments and/or further referrals are required.

To sum up, embodiments of the disclosure provide a way to present a VR scene for a subject, and to ask the subject to carry out a number of assignments that fit an intended scene as defined in a questionnaire, so as to enable the subject to give more realistic response to the assignments. Additionally, a sequence in which the assignments are asked to be performed by the subject may be subject-specific based on the responses of the subject to previous assignments.

In such a virtually created environment, the responses (including the inputs of the subject, head movement, eye movement, HR distribution, etc.) recorded by the recording device 4 may be more objective then simply requesting the subject to answer the questionnaire since the subject may not always give the most accurate answer. Using such a manner as described above, a more accurate assessment for specific mental and behavior conditions may be carried out for the subject, and it may be possible to detect less severe syndromes that are typically not detectable using the questionnaire alone.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding various inventive aspects.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for performing an assessment of mental and behavioral condition of a subject, the method comprising steps of:
displaying, by a virtual reality (VR) device, a VR scene;
generating, by the VR device, a notification indicating an assignment for the subject in the VR scene;
recording, by a recording device, a response of the subject to the assignment;
calculating, by a processing device, at least one score based on the response; and
comparing, by the processing device, the score with a pre-established normative scale to generate an assessment result for the mental and behavioral condition of the subject.

2. The method of claim 1, further comprising steps of:
detecting, by a detecting subsystem, an attended-to position in the VR scene where the subject pays attention to during execution of the assignment; and
recording, by the recording device, a degree of completion of the assignment, the response and the attended-to position in an assignment record associated with the assignment,
wherein the step of calculating is to calculate the score based on the assignment record.

3. The method of claim 2, further comprising, prior to the step of calculating, steps of:
determining, by the processing device, a follow-up assignment based on the assignment record;
controlling, by the processing device, the VR device to further generate at least one notification indicating the follow-up assignment for the subject; and
repeating the steps of receiving, detecting and recording for the follow-up assignment so as to generate another assignment record associated with the follow-up assignment,
wherein the step of calculating is implemented when one of a first condition that a predetermined number of assignments has been executed by the subject and a second condition that a predetermined length of time has elapsed is satisfied, and is to calculate the score based on the assignment records.

4. The method of claim 2, the detecting subsystem including an eye tracking device for detecting a point of gaze of the subject as the attended-to position,
wherein the method further comprises steps of constructing, by the recording device, an attention circle with the point of gaze as a center and determining, by the recording device, a movement and coverage of the attention circle in the VR scene, and the step of recording is to further record the movement and coverage of the attention circle in the assignment record.

5. The method of claim 2, further comprising, prior to the step of calculating, a step of generating, by the VR device, at least one auxiliary notification in the VR scene for drawing attention of the subject during the execution of the assignment by the subject.

6. The method of claim 5, the detecting subsystem including an eye tracking device for detecting a point of gaze of the subject as the attended-to position, wherein:
the method further comprises steps of constructing, by the recording device, an attention circle with the point of gaze as a center and determining, by the recording device, a movement and coverage of the attention circle in the VR scene, and the step of recording is to further record the movement and coverage of the attention circle in the assignment record.

7. The method of claim 2, wherein the step of recording is to further record, in the assignment record, a length of time taken by the subject to complete the assignment.

8. The method of claim 2, the detecting subsystem including a heart rate measuring device, wherein the step of recording is to further record a heart rate distribution of the subject in a period of time in which the subject undertakes the assignment, in the assignment record.

9. The method of claim 8, further comprising, prior to the step of calculating, a step of generating, by the VR device, at least one auxiliary notification in the VR scene for drawing attention of the subject during the execution of the assignment by the subject.

10. The method of claim 1, wherein the response recorded in the step of recording comes from at least one of an input subsystem operated by the subject and a detecting subsystem that detects an attended-to position in the VR scene where the subject pays attention to during execution of the assignment.

11. A system for performing an assessment of a mental and behavioral condition for a subject, comprising:
a virtual reality (VR) device;
a recording device; and
a processing device communicating with said VR device and said recording device, and being programmed to
control said VR device to display a VR scene, and to generate a notification in the VR scene, the notification indicating an assignment for the subject in the VR scene,
in response to said recording device recording a response of the subject to the assignment, calculating at least one score based on the response, and
comparing the score with a pre-established normative scale to generate an assessment result for the mental and behavioral condition of the subject.

12. The system of claim 11, further comprising a detecting subsystem that is programmed to detect an attended-to position in the VR scene where the subject pays attention to during execution of the assignment;
wherein said recording device is further programmed to record a degree of completion of the assignment, the response and the attended-to position in an assignment record that is associated with the assignment,
wherein said processing device is programmed to calculate the score based on the assignment record.

13. The system of claim 12, wherein said processing device is further programmed to, prior to calculating the score:
determine a follow-up assignment based on the assignment record;
control said VR device to further generate at least one notification indicating the follow-up assignment for the subject; and
repeat the receiving, detecting and recording for the follow-up assignment so as to generate another assignment record associated with the follow-up assignment,
wherein said processing device is programmed to implement the calculating of the score when one of a first condition that a predetermined number of assignments has been executed by the subject and a second condition that a predetermined length of time has elapsed is satisfied, and is to calculate the score based on the assignment records.

14. The system of claim 12, wherein:
said detecting subsystem includes an eye tracking device for detecting a point of gaze of the subject as the attended-to position; and
said recording device is programmed to construct an attention circle with the point of gaze as a center, determine a movement and coverage of the attention circle in the VR scene, and further record the movement and coverage of the attention circle in the assignment record.

15. The system of claim 12, wherein, prior to calculating the score, said processing device further controls said VR device to generate at least one auxiliary notification in the VR scene for drawing attention of the subject during the execution of the assignment by the subject.

16. The system of claim 15, wherein:
said detecting subsystem including an eye tracking device for detecting a point of gaze of the subject as the attended-to position; and
said recording device is programmed to construct an attention circle with the point of gaze as a center, determine a movement and coverage of the attention circle in the VR scene, and further record the movement and coverage of the attention circle in the assignment record.

17. The system of claim 12, wherein said recording device is further programmed to record, in the assignment record, a length of time taken by the subject to complete the assignment.

18. The system of claim 12, the detecting subsystem including a heart rate measuring device, wherein the step of recording is to further record a heart rate distribution of the subject in a period of time the subject undertakes the assignment, in the assignment record.

19. The system of claim 18, wherein, prior to calculating the score, said processing device further controls said VR device to generate at least one auxiliary notification in the VR scene for drawing attention of the subject during the execution of the assignment by the subject.

20. The system of claim 11, wherein said recording device records the response from at least one of an input subsystem operated by the subject and a detecting subsystem that detects an attended-to position in the VR scene where the subject pays attention to during execution of the assignment.
